(19) United States
(12) Patent Application Publication
(10) Pub. No.: US 2019/0374477 A1
(43) Pub. Date: Dec. 12, 2019

(54) EYE-INJECTABLE POLYMERIC NANOPARTICLES AND METHOD OF USE THEREFOR

(71) Applicants: FONDAZIONE ISTITUTO ITALIANO DI TECNOLOGIA, GENOVA (IT); ISTITUTO DON CALABRIA - OSPEDALE CLASSIFICATO SACRO CUORE, NEGRAR (IT)

(72) Inventors: Guglielmo LANZANI, MILANO (IT); Fabio BENFENATI, GENOVA (IT); Grazia PERTILE, TORRI DEL BENACO (IT); Maurizio METE, VERONA (IT); José Fernando MAYA-VETENCOURT, PISA (IT)

(21) Appl. No.: 16/005,248

(22) Filed: Jun. 11, 2018

(51) Int. Cl. A61K 9/51; A61K 31/795; A61K 9/00; A61K 41/00; A61P 27/02

(52) U.S. Cl. CPC A61K 9/51; A61K 31/795; A61P 27/02; A61K 9/0019; A61K 41/0042; A61K 9/0048

(57) ABSTRACT

The present invention refers to a method for treating visual deficits comprising at least one step of injecting in the eye of a subject in need thereof a therapeutically effective amount of photoactive nanoparticles (NPs) or a composition comprising said photoactive nanoparticles (NPs).

### TECHNICAL FIELD

The present invention refers to a method for treating visual deficits comprising at least one step of injecting in the eye of a subject in need thereof a therapeutically effective amount of photoactive nanoparticles (NPs) or a composition comprising said photoactive nanoparticles (NPs).

### BACKGROUND ART

Millions of people around the world suffer from ocular pathologies. In particular, retinal degenerative diseases are debilitating conditions with a major impact on daily life. From performing basic functions to personal independence and mental health, vision loss affects a wide range of everyday tasks such as reading, watching TV, driving etc.

Retinal degenerative diseases are associated with damages against photoreceptor cells of the retina. Photoreceptors are cells that begin the process of seeing by absorbing and converting light into electrical signals. These signals are sent to other cells in the retina and ultimately through the optic nerve to the brain where they are processed into the images we see. There are two general types of photoreceptors, called rods and cones. Rods are in the outer regions of the retina and allow us to see in dim and dark light. Cones reside mostly in the central portion of the retina and allow us to perceive fine visual detail and color.

When photoreceptor cells malfunction because of a degenerative disease, the received image results blurred, distorted or completely unseen. The degenerative process is progressive as well as the related decline in vision.

The most common retinal degenerative diseases are Age-related Macular Degeneration and Retinitis Pigmentosa.

Retinitis Pigmentosa (RP) is caused by mutations in a large array of genes involved in phototransduction and consists in a primary degeneration of rods with late involvement of cone survival. Accordingly, scotopic vision is precociously affected in RP patients, with a progression toward total blindness. Scotopic vision means the vision of the eye under low-light levels. Because, cone cells are nonfunctional in low visible light, scotopic vision is produced exclusively through rod cells, which are most sensitive to wavelengths of light around 498 nm (green-blue) and are insensitive to wavelengths longer than about 640 nm (red).

Age-related macular degeneration (AMD) consists of a selective degeneration of foveal cones and affected a high percentage (up to 20%) of people above 75 years of age. In AMD, foveal photoreceptors are primarily impaired, resulting in loss of high-resolution vision in the central area of the visual field. Fovea (also called central fovea or fovea centralis) is a tiny pit located in the macula of the retina that provides the clearest vision of all. Only in the fovea are the layers of the retina spread aside to let light fall directly on the cones, the cells that give the sharpest image.

In spite of the very high prevalence of AMD, the therapeutic approaches to the vast majority of AMD cases (dry or atrophic AMD) have been unsatisfactory and no approved clinical treatment exists at the moment. Wet forms of AMD can be effectively controlled by anti-VEGF medications or the subfoveal auto-transplant of a Retinal Pigmented Epithelium (RPE) patch. However, no surgical interventions are suited for dry AMD. Indeed, with the exception of new experimental therapies, such as the subretinal transplantation of stem cells to regenerate RPE cells and, possibly, photoreceptors, no promising perspectives exist.

Retinal prostheses generating light-induced electrical signals in the retina represent an alternative approach for sight restoration in retinopathies with photoreceptor degeneration. Several groups in the world are currently working on retinal prosthesis, some of which are already in the stage of human testing. The major common problems so far encountered by these metal/silicon devices are: need of power supply, scarce biocompatibility, poor contact with the tissue due to the rigidity of the device and motion of the eye, electrode size and limited number of pixels, high impedance levels and pronounced heat production.

Moreover, as mentioned above, foveal cones, the smallest photoreceptors present in the retina, which display the densest wiring to the optic nerve and the largest cortical representation, selectively degenerate in AMD, resulting in loss of vision in the central visual field associated with a sharp decrease in visual acuity. In this regard, currently, no artificial prostheses fulfill the required spatial resolution requirements needed to restore the high-resolution central vision lost in AMD patients.

In view of the above, it would be particularly desirable to provide a strategy for treating visual deficits, preferably associated with degeneration of photoreceptors, such as RP and AMD, and to restore a high-resolution sight. In particular, the need to obtain alternative approaches to guarantee recovery of foveal cones functionalities is strongly felt.

The solution here proposed involves eye's injection of photoactive nanoparticles (NPs), in particular used as aqueous suspension. Advantageously, the injection of the NPs here disclosed requires a simpler surgical operation with respect to planar, finite-size prosthesis. Moreover, the injected NPs are fully dispersed in the biological tissue, providing better interfacing and resolution with respect to a monolayer of NPs on a substrate that would be similar to a planar prosthesis. The particle size hampers dangerous cell internalization yet provides high resolution and efficient coupling with the neuronal layers through nanoparticle surface. In addition, contrary to inorganic, metal nanoparticles, the NPs of the present invention (I) do not leach toxic ions; (II) do not cause tissue heating; (III) due to their specific photoexcitation dynamics, do not generate singlet oxygen that may lead to cell apoptosis. A further advantage of using the NPs in the disclosed context is related to their negative surface charge (Z potential around −40 mV) that provide with stability and cell affinity.

### SUMMARY OF THE INVENTION

The present invention refers to a method for treating a visual deficit of at least one eye of a subject comprising at least one step of injecting in the eye of said subject a therapeutically effective amount of photoactive nanoparticles (NPs) or a composition comprising said photoactive nanoparticles (NPs). In particular, the invention refers to a method for improving the spatial resolution of at least one eye of a subject comprising at least one step of injecting in the eye of said subject a therapeutically effective amount of photoactive nanoparticles (NPs), preferably an aqueous dispersion of NPs, preferably the NPs comprise poly-(3-hexylthiophene), in other words are P3HT-NPs.

The NPs or the composition comprising NPs is/are preferably administered intraorbitally or intracapsular, preferably by microinjection into a subretinal space.

The visual deficit is preferably associated with a condition selected from: Retinitis Pigmentosa (RP) or related syndromes, preferably said syndromes being selected from: Usher Syndrome, Bardet-Biedl syndrome, Refsum disease, Batten disease, and Jalili syndrome; neuropathy, ataxia, NARP (neuropathy, ataxia, and retinitis pigmentosa) syndrome, inherited retinal degenerations, preferably elected from: Stargardt's disease and Leber's congenital amaurosis, and atrophic age-related macular degeneration (AMD).

The present invention refers also to a kit for performing the method comprising the photoactive NPs or a composition comprising said NPs.

### BRIEF DESCRIPTION OF DRAWINGS

FIG. 1 shows the anatomy of the human eye. The eye is a fluid-filled sphere enclosed by three layers of tissue. Only the innermost nervous part of the eye, the retina, contains three layers of neurons of which the most external one is light-sensitive—the photoreceptors. Light is converted by photoreceptors into electrical activation/inhibition of the intermediate layer of neurons (bipolar cells); the information is preprocessed by the retinal circuits and eventually conveyed to the innermost layer (retinal ganglion cells) that transmits visual signals to the central nervous system. The immediately adjacent layer of tissue is the choroid, which is composed of a rich capillary bed (important for nourishing photoreceptors in the retina) as well as a high concentration of the light absorbing pigment melanin. The iris is the colored portion of the eye that can be seen through the cornea. It contains two sets of muscles with opposing actions, which allow the size of the pupil to be adjusted under neural control. The sclera forms the outermost tissue layer of the eye and is composed of an opaque fibrous tissue. At the front of the eye, however, this opaque outer layer is transformed into the cornea, a specialized transparent tissue that permits light rays to enter the eye. Beyond the cornea, light rays pass through two distinct fluid environments and strike the retina. The space between the back of the lens and the surface of the retina is filled with a thick, gelatinous substance called vitreous humor, which accounts for about 80% of the eye volume. In addition to maintaining the shape of the eye, vitreous humor, contains phagocytic cells that remove blood and other debris that might otherwise interfere with light transmission.

FIG. 2 shows: (a) a general scheme of the procedure to produce P3HT-NPs by using precipitation and differential centrifugation methods. (b) Dynamic Light Scattering (DLS) and zeta-potential data of two different subpopulations of P3HT-NPs of average diameter 148±18 nm (left panel) and 344±60 nm (right panel), calculated over 3 independent measurements. (c) Scanning Electron Microscopy (SEM) images of the 148 nm and 344 nm sized NPs (left and right panels, respectively). Scale bar, 500 nm.

FIG. 3 shows confocal Scanning Laser Ophthalmoscopy (cSLO) images of the dystrophic Royal College of Surgeons (RCS) rats retina one month after the subretinal microinjection of either glass NPs and P3HT NPs (left) and Ocular Coherence Tomography (OCT) analysis showing the integrity of the entire retina, in the absence of retinal detachments or breakages (right).

FIG. 4 shows the pupillary reflex of rats at 30-day post-injection (DPI) assessed using standard infrared imaging in implanted RCS. Extent of the PLR as a function of the light intensity, showing the P3HT NP-induced recovery of light sensitivity in RCS rats at 30 DPI at intensity >2 lux with respect to the control glass NPs.

FIG. 5 shows a behavioral evaluation of visual function of rats injected with P3HT NPs. The light-dark box test revealed the reinstatement of light sensitivity in RCS rats injected with P3HT NPs, but not glass NPs, evaluated as both escape latency from the lit compartment (upper panel) and time spent in the dark compartment (middle panel) at 30 DPI. The locomotor activity of the four experimental groups was assessed by measuring the number of light-dark transitions (lower panel).

FIG. 6 shows an electrophysiological assessment of cortical visual responses in response to flash and patterned illumination. Upper panel VEP recordings in V1 (a) in response to flash stimuli (20 cd m⁻²; 100 ms) show the rescue of light sensitivity in P3HT-NP injected RCS rats at 30 days post-injection (DPI). RCS rats that were sham-injected with glass NPs had no visual improvement. Lower panel: the electrophysiological analysis of VEP recordings in response to horizontal sinusoidal gratings of increasing spatial frequencies (0.1 to cycle/degree of visual angle) administered at 0.5 Hz reveals a significant recovery of visual activity at 30 DPI. RCS rats that were sham-injected with glass NPs had no visual improvement.

### DEFINITIONS

As used herein, “visual deficit” means an impairment of both visual acuity and binocularity.

As used herein, “blindness” means a disease state of low or absent vision, preferably said blindness is secondary to the degeneration of retinal rods and/or cone photoreceptors.

As used herein, “spatial resolution” means the ability of visual cortical neurons to distinguish two points in the visual field, each one as different from the other.

As used herein, “high sensitivity” of the eye means the ability of our retina to perceive the visual field in ambient light as well as at low luminance levels.

As used herein, “visual performance” means the ability to perceive visual information with high spatial resolution and contrast sensitivity.

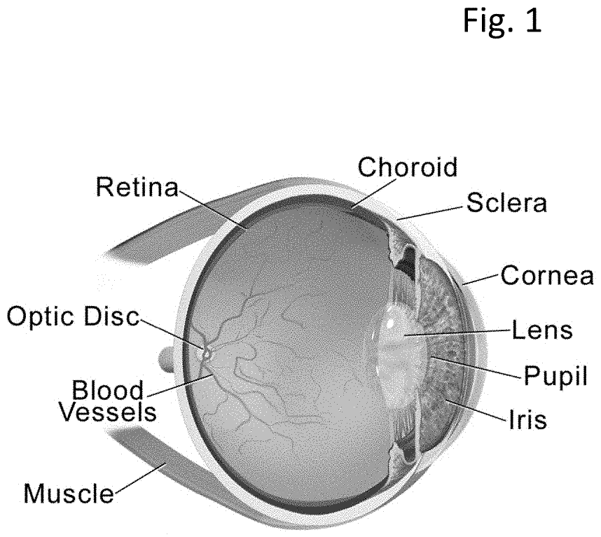

As used herein, the term “eye” refers to an organ of the visual system as illustrated in FIG. 1. It provides organisms with vision, the ability to receive and process visual detail, as well as enabling several photo response functions that are independent of vision. The innermost nervous layer of the eye, the retina, contains light-sensitive cells (see FIG. 1) that convert the energy carried by photons into electro-chemical impulses that are transmitted to the brain (see FIG. 1).

As used herein, the term “photoreceptors” refers to the cells that begin the process of seeing by absorbing and converting light into electrical signals. The resulting electrical activation/inhibition of the intermediate layer of neurons (bipolar cells) that is preprocessed by the retinal circuits is conveyed to the innermost layer (retinal ganglion cells) that transmit the visual information to the central nervous system through the optic nerve. These signals are sent to other cells in the retina and ultimately through the optic nerve to the brain where they are processed into the images we see. There are two general types of photoreceptors, called rods and cones. Rods are in the outer regions of the retina and allow us to see in dim and dark light. Cones reside mostly in the central portion of the retina and allow us to perceive fine visual detail and color.

As used herein, the term “nanoparticles” (NPs) refers to polymer aggregates having preferably approximate spherical shape of radius preferably between 30 and 600 nm. Moreover, said NPs are preferably stable in water suspension and injectable in the subretinal space where they absorb light and transduce it into inner retina cells stimulation. Light induces charge separation that negatively charges the particle surface interacting with retinal neurons and causes electrical and biochemical alterations of the electrical state of bipolar cells.

As used herein, the terms “treatment” or “treating” refer to the medical management of a subject with the intent to cure, ameliorate, stabilize, or prevent a condition or disorder. Therefore, in the contest of the present invention treating means also rescuing or ameliorating or curing.

As used herein, the term “applying” refers to any method of providing or administering a composition and/or pharmaceutical composition thereof to a subject, preferably applying by microinjection into the subretinal space. The path to the subretinal space could be through the corpus vitreous, or through a less invasive scleral flap.

As used herein, the term “therapeutically effective amount” refers to an amount that is sufficient to achieve the desired therapeutic result or to have an effect on undesired symptoms but is generally insufficient to cause adverse side effects.

As used herein, the terms “subject” or “subject in need thereof” refer to a target of administration, which optionally displays symptoms related to a particular disease, condition, disorder, or the like. The subject(s) (or individual) of the herein disclosed methods can be human or non-human (e.g., primate, horse, pig, rabbit, dog, sheep, goat, cow, cat, guinea pig, rodent, and non-mammals).

As used herein, the term “retina” refers to the innermost coat of the eye, which is a light-sensitive layer of tissue (see FIG. 1).

As used herein, the term “sclera” refers to the opaque, fibrous, protective, outermost layer of the eye containing mainly collagen and some elastic fiber (see FIG. 1).

As used herein, the term “choroid” refers to the vascular layer of the eye, containing connective tissues, and lying between the retina and the sclera. Overlooking the external segments of photoreceptors, the choroid is covered by the Retinal Pigment Epithelium (RPE) that feeds photoreceptors and is necessary for their survival and turnover. Neural sampling of retinal images by the cone photoreceptor mosaic in the fovea is fundamental to define visual resolution. Individual foveal cones connect via a cone bipolar cell to at least two ON/OFF retinal ganglion cells of the “private line” midget system in the central retina. As a result, the fovea is responsible of almost all our photopic information and, accordingly, provides the vast majority of the retinal input to the visual cortex.

As used herein, the term “fovea” refers to a small, central pit composed of closely packed cones in the eye. It is located in the center of the macula lutea of the retina.

As used herein, the term “hydrodynamic diameter” refers to the nanoparticle diameter measured by a light scattering apparatus. This value is a standard figure of merit for the particle size that however exceeds the physical particle size, as measured for instance by transmission electron microscopy (TEM). The difference is due to a surface layer covering the particle and to the polarization of the medium.

As used herein, the term “Z-potential” refers to the potential difference between the dispersion medium and the stationary layer of fluid attached to the dispersed particle.

As used herein, the term “aqueous environment” refers to a solvent or dispersion medium made primarily by water.

As used herein, the term “polydispersity index” (PDI) refers to a standard figure of merit characterizing the particle size distribution. It is calculated by taking the size distribution of a sample and dividing its standard deviation by its mean.

As used herein, the term “microinjection” refers to the administration of a substance using a microsyringe in order to slowly and regularly inject microliter volumes at microscopic level.

As used herein, the term “retinal degeneration” refers to the apoptotic degeneration of photoreceptors in the retina, a phenomenon that eventually leads to total blindness.

As used herein, the term “subretinal space” or “subretinal region” refers to the area between the retina pigment epithelium (RPE) and the bipolar cell layer, once the photoreceptor layer has disappeared because of degeneration.

As used herein, the term “visible range” refers to the portion of the electromagnetic spectrum that is visible to the human eye, in the wavelengths from about 390 to 700 nm.

### DETAILED DESCRIPTION OF PREFERRED EMBODIMENTS OF THE INVENTION

A first aspect of the present invention refers to a method for treating a visual deficit in a subject in need thereof comprising at least one step of applying to the eye of said subject a therapeutically effective amount of photoactive nanoparticles (NPs) or a composition comprising photoactive nanoparticles (NPs).

Preferably, the method of the disclosure allows completely or at least partially curing or rescuing said visual deficit or even ameliorating said visual deficit in the subject in need thereof.

The photoactive nanoparticles (NPs) or a composition comprising photoactive nanoparticles (NPs) as here disclosed can be also useful to rescue high sensitivity and/or visual performances of a malfunctioning eye of the subject in need thereof.

A further aspect of the present invention refers to a method for improving the spatial resolution as defined above of at least one eye of a subject in need thereof comprising at least one step of applying to the eye of said subject a therapeutically effective amount of photoactive nanoparticles (NPs) or a composition comprising photoactive nanoparticles (NPs).

A further aspect of the present invention refers to a method for treating blindness, preferably secondary to the degeneration and/or malfunction of eye photoreceptors in a subject in need thereof comprising at least one step of applying to the eye of said subject a therapeutically effective amount of photoactive nanoparticles (NPs) or a composition comprising photoactive nanoparticles (NPs).

In the contest of the present invention the term amount means also number, therefore the expression “amount of photoactive nanoparticles” means also “number(s) of photoactive nanoparticles”.

Therefore, the photoactive nanoparticles (NPs) or a composition comprising photoactive nanoparticles (NPs) as here disclosed can be also defined or considered a substitute of degenerated and/or impaired photoreceptors of the eye.

Preferably, the NPs comprise poly-(3-hexylthiophene), more preferably said NPs are made of poly-(3-hexylthiophene), named P3HT-NPs.

According to the preferred embodiment of the invention, the NPs are made by a conjugated carbon-based polymer. In other words, the NPs show/have a conjugated carbon-based polymer. This feature allows the NPs have optical absorption in the visible spectral range. This implies an effective conjugation length allowing sufficient electron delocalization to yield an electronic resonance in the visible spectrum, preferably with peak around 2.4 eV. In this context effective conjugation length means the average size of the conjugated segment in between conjugation breaks.

According to a preferred embodiment, the NPs, preferably the P3HT-NPs, are used as aqueous preparation/solution, preferably an aqueous dispersion or suspension of NPs/P3HT-NPs.

According to a preferred embodiment, the NPs, preferably the P3HT-NPs are characterized by a diameter ranging from 50 to 450 nm, preferably from 75 to 400 nm, more preferably from 90 to 350 nm. Said diameter is preferably a hydrodynamic diameter (HD) as defined above.

In a preferred embodiment, the NPs, preferably the P3HT-NPs, are characterized by a diameter of about 148 nm or about 344 nm.

According to a preferred embodiment, the NPs, preferably the P3HT-NPs, are characterized by a polydispersity index (PDI) comprised between 0.008 and 0.05, preferably between 0.009 and 0.04, more preferably between 0.01 and 0.03.

In a preferred embodiment, the NPs, preferably the P3HT-NPs, are characterized by PDI of about 0.015, preferably for the NPs having diameter of about 148 nm and PDI of about 0.03, preferably for the NPs having diameter of about 344 nm.

According to a preferred embodiment, the NPs, preferably the P3HT-NPs, are characterized by a Z-potential value less or equal to −30 mV. In a preferred embodiment the NPs, preferably the P3HT-NPs have a Z-potential value of about −30.44 mV, preferably for the NPs having diameter of about 148 nm, and −36.84 mV, preferably for the NPs having diameter of about 344 nm.

According to a preferred embodiment, the NPs, preferably the P3HT-NPs, absorb the wavelength of visible light, preferably a wavelength ranging from 450 to 650 nm, more preferably from 460 to 640 nm, still more preferably from 495 to 620 nm.

In a preferred embodiment, the NPs, preferably the P3HT-NPs, absorb the light with a peak in the green-orange region depending on the size. More preferably NPs/P3HT-NPs having diameter of about 148 absorb the light from 495 to 570 nm, while NPs/P3HT-NPs having diameter of about 344 absorb the light from 590 to 620 nm.

According to a preferred embodiment, the NPs, preferably the P3HT-NPs, are preformed NPs/P3HT-NPs and modified as disclosed below. Alternatively said NPs/P3HT-NPs are prepared/obtained preferably by using the procedure disclosed in G. Barbarella et al, 1994.

The procedure to prepare the P3HT-NPs of the invention comprises preferably the following steps:

(i) Providing 3-hexylthiophene (3HT);

(ii) Obtaining poly-3-hexylthiophene (P3HT) by chemical polymerization of the 3HT of step (i), preferably 1 gram in 40 mL of CHCl₃, with preferably dry FeCl₃. Solid FeCl₃ (dry) is preferably used in the following amount: 3.87 g in suspension in 100 mL of CHCl₃.

Alternatively, step (i) and (ii) can be skipped providing NPs/P3HT-NPs having a solubility preferably over 0.02 g/L in the solvent used for the reprecipitation, more preferably a M_{w}<90000 and/or Mₙ<50000.

Step (ii) is preferably performed by stirring the reaction mixture overnight, preferably at speed greater than 60 rpm, preferably at room temperature and preferably then worked up with an alcohol, preferably MeOH.

According to a preferred embodiment, the polymer is subsequently extracted preferably with a Soxhlet apparatus, preferably at least one night by using methanol, and/or at least one night by using acetone.

The obtained solid is preferably dried, preferably under vacuum and eventually subsequently washed, preferably by using a hydrazine solution, preferably in a concentration 2% w/w in water.

The aqueous phase is preferably filtered, and the solid is dissolved preferably in a solvent, more preferably THF. Subsequently the solid is precipitated again by using an alcohol, preferably MeOH.

According to a preferred embodiment, the NPs/P3HT-NPs are made by the nano-precipitation method that preferably comprises the following steps.

The starting material (P3HT) is first dissolved in a solvent, preferably in tetrahydrofuran, paying attention to maintain their sterility. This solution is injected into a sterile non-solvent for the polymer that is preferably miscible with the used solvent, preferably water. Alternatively, the sterile non-solvent can be injected into the polymer solution. The injection is preferably done under magnetic stirring and/or ultrasound assistance in sterile environment. The rapid change in solvent polarity favors p-p stacking and hydrophobic interactions determining the formation of nanoparticles. However, while in mini-emulsification—a further common method for preparation of nanoparticles—the organic solvent used to dissolve the starting material is immiscible with water and requires the presence of surfactants, preferably sodium dodecyl sulfate, to avoid coalescence of the emulsified droplets, in nano-precipitation the organic solvent is miscible with water and the resulting NP dispersion is free of added surfactants.

Generally, the nano-precipitation method yields smaller nanoparticles as compared to mini-emulsification. In general, the preparation method and experimental conditions, preferably the organic solvent and/or the mixture of solvents, the starting material concentration, the polydispersity and regio-regularity of the polymer, the stirring speed, etc, strongly affect the size and the internal supramolecular organization of the material in the NPs/P3HT-NPs, which are in turn intimately related to their optoelectronic, charge transfer and charge transport properties.

The NPs, preferably the P3HT-NPs, are then selected under sterility conditions, preferably by centrifuging, more preferably by using differential centrifugation, based on their diameter, preferably the hydrodynamic diameter and/or by the polydispersity index as disclosed and defined in detail above. If the solvent used is not biocompatible, the NPs dispersion is dialyzed preferably against physiological solution or sterile water that can be injected subretinally.

According to a preferred embodiment, the selected NPs/P3HT-NPs are subsequently linked, preferably by a covalent bond, with ionic side groups, preferably by grafting on the hydroxyl side groups, and/or by encapsulating the NPs/P3HT-NPs with micelles, and/or by loading the NPs with polyethylene glycol (PEG). Surface functionalization can improve particle chemical-physical properties by reducing their tendency to coalesce and improving their stability, as well as favoring the adhesion of particles to specific tissues or cell membranes.

Therefore, according to a further embodiment of the disclosure before being administered the poly-(3-hexylthiophene) NPs are treated according to the following steps:

a) Collecting poly-(3-hexylthiophene), preferably by centrifugation; and/or

b) Selecting NPs based on different hydrodynamic diameter and/or on polydispersity index; and/or

c) Suspending the NPs in aqueous solution; and/or

d) Linking covalently the NPs with ionic side groups; and/or

e) Grafting on hydroxyl side groups.

According to a preferred embodiment, said hydrodynamic diameter ranges between 50 and 450 nm, more preferably between 75 and 400 nm, still more preferably between 90 and 350 nm.

In a preferred embodiment, the hydrodynamic diameter is about 148 nm and/or about 344 nm.

According to a preferred embodiment, said polydispersity index (PDI) ranges between 0.008 and 0.05, more preferably between 0.009 and 0.04, still more preferably between 0.01 and 0.03.

Preferably the treated poly-(3-hexylthiophene) NPs are then encapsulated with micelles and/or dispersed in polyethylene glycol (PEG).

According to a preferred embodiment, the visual deficit is associated with degenerated photoreceptors in the eye, preferably with cones, more preferably foveal cones.

In this context degeneration means the selective degeneration of photoreceptors, preferably cones that is generally typical of retinal degenerative diseases preferably macular degeneration.

According to a preferred embodiment, the degeneration is associated with a condition selected from: Retinitis Pigmentosa (RP) or related syndromes, preferably said syndromes being selected from: Usher Syndrome, Bardet-Biedl syndrome, Refsum disease, Batten disease, and Jalili syndrome; neuropathy, ataxia, NARP (neuropathy, ataxia, and retinitis pigmentosa) syndrome, inherited retinal degenerations, preferably elected from: Stargardt's disease and Leber's congenital amaurosis, and atrophic age-related macular degeneration (AMD).

Therefore a further aspect of the invention refers to a method for treating in a subject an ocular disorder in which photoreceptor degeneration, preferably primary photoreceptor degeneration is involved with preservation of an inner retinal layer, preferably selected from: Retinitis Pigmentosa (RP) or related syndromes, preferably said syndromes being selected from: Usher Syndrome, Bardet-Biedl syndrome, Refsum disease, Batten disease, and Jalili syndrome; neuropathy, ataxia, NARP (neuropathy, ataxia, and retinitis pigmentosa) syndrome, inherited retinal degenerations, preferably elected from: Stargardt's disease and Leber's congenital amaurosis, and atrophic age-related macular degeneration (AMD), said method comprising at least one step of applying to the eye of said subject a therapeutically effective amount of photoactive NPs/P3HT-NPs or a composition comprising photoactive NPs/P3HT-NPs as disclosed above.

Preferably said method is particularly effective in treating Retinitis Pigmentosa and/or AMD, preferably dry AMD.

Preferably, the method is for rescuing high sensitivity of degenerated retina.

Preferably, said NPs, preferably the P3HT-NPs, or said composition comprising NPs/P3HT-NPs are administered subretinally by microinjection. Said microinjection is preferably performed through the sclera and/or through the vitreous chamber.

According to a preferred embodiment the NPs, preferably the P3HT-NPs or the composition comprising said NPs/P3HT-NPs, are(is) administered/applied/injected, preferably by microinjection, into the subretinal space.

Preferably, the method involves the following steps:

(i) Performing an opening (cut/incision) on the conjunctiva of the eye to be treated, preferably with scissors, starting 1.5 mm from the limbus for about 2 clock hours in the upper quadrants; and/or

(ii) Incising the sclera and the choroid—about 0.6 mm—preferably 1 mm from the limbus; and/or

(iii) Separating the retina from the retinal pigment epithelium by injecting a small amount of a viscoelastic material into the subretinal space; and/or

(iv) Injecting the NPs/P3HT-NPs or the composition comprising said NPs/P3HT-NPs, preferably through to the sclera, into the sub-retinal space; and/or

(v) Coagulating the scleral incision preferably by diathermy and preferably repositioning the conjunctiva over the scleral wound.

In this context, viscoelastic material is used for its properties to prevent mechanical damage to tissue, provide a wider space for surgical manipulation, and avoid adhesions postoperatively. Preferably said viscoelastic material is selected from: hyaluronic acid sodium salt, hydroxypropylmethylcellulose or chondroitin sulfate, preferably high molecular weight hyaluronic acid sodium salt.

In one embodiment, the NPs, preferably the P3HT-NPs, or the composition comprising said NPs/P3HT-NPs are administered by microinjection at the macula by first penetrating the sclera, or by microinjection into the sub-retinal region.

According to a preferred embodiment, the injection is performed by opening the conjunctiva, incising the sclera and the choroid, separating the sclera and the retinal pigment epithelium, injecting a viscoelastic material as defined above into the retina and finally injecting the NPs or the composition comprising the NPs/P3HT-NPs in a subretinal region.

According to a further preferred embodiment, the NPs, preferably the P3HT-NPs or the composition comprising said NPs/P3HT-NPs are injected tangentially to the sclera, in order to prevent any damage of the retina and the choroid. The tangential sub-retinal flow originated injecting with the needle in this position is very efficient in promoting a complete retinal detachment and a consequent uniform distribution of the nanomaterial.

A second aspect of the present application refers to a composition comprising the NPs, preferably the P3HT-NPs, said NPs/P3HT-NPs being characterized according to the detailed disclosure reported above.

The composition is preferably an ophthalmic pharmaceutical composition, more preferably an injectable ophthalmic composition.

The ophthalmic composition of the present invention is characterized by a pH generally acceptable for ophthalmic applications. Preferably, the pH value of the composition ranges between 7.0 and 7.5.

Moreover, the composition is characterized by an osmotic pressure generally acceptable for ophthalmic applications. Preferably, the osmotic pressure value of the composition ranges between 290 and 300 mOsm/L.

Eventually, the composition comprises a pharmaceutically effective amount of an additional ingredient preferably selected from: anti-inflammatory, astringent drugs, preferably selected from: neostigmine methylsulfate, ε-amino caproic acid, allantoin, berberine chloride, zinc sulfate, lysozyme chloride, sodium azulene sulfonate, dipotassium glycyrrhizinate and combinations thereof; antiallergic agents, preferably selected from: diphenhydramine hydrochloride, isopenzyl hydrochloride, chlorpheniramine maleate, sodium cromoglycate and combinations thereof; vitamins other than pyridoxine hydrochloride, preferably selected from: vitamin B2, vitamin B12, vitamin A, vitamin E, calcium pantothenate and combinations thereof, amino acids, preferably selected from: potassium L-aspartate, magnesium L-aspartate, aminoethylsulfonic acid and combinations thereof; sulfa drugs, preferably selected from: sulfamethoxazole, sulfisoxazole, sulfisomidine and combinations thereof, bacteriocides, preferably selected from: sulfur, isopropylmethyl phenol, hinokithiol and combinations thereof; topical anesthetics, preferably selected from: lidocaine, lidocaine hydrochloride, procaine hydrochloride, dibucaine hydrochloride and combinations thereof, inorganic salts, preferably selected from: potassium chloride, sodium chloride, sodium bicarbonate and combinations thereof, thickening agents, preferably selected from: polyvinyl alcohol, polyvinyl pyrrolidone, carboxymethyl cellulose, hyaluronic acid, glucose and combinations thereof.

According to a further embodiment, the composition comprises a variety of additives, preferably solubilization auxiliary agents, isotonizing agents, stabilizing agents, chelating agents, pH adjusting agents, refrigerants, preservatives, and thickening agents; and/or
a carrier, preferably buffer agents or ointment bases, which may be generally used in ophthalmic compositions can also be blended.

The composition of the present invention can be for any formulation generally used in ophthalmic compositions, preferably selected from: aqueous solutions, aqueous suspensions, aqueous dispersion, emulsions, gelatinous materials, ointments and combinations thereof.

In a preferred embodiment, the composition is formulated as aqueous solution/aqueous suspension/aqueous dispersion suitable for being injected, in particular suitable for being injected in a subretinal region.

Another aspect of the present invention refers to a kit comprising the NPs/P3HT-NPs or the composition of the present invention. In a preferred embodiment, the kit is used for medical applications, in particular in the ophthalmic fields.

### Examples

NPs Preparation

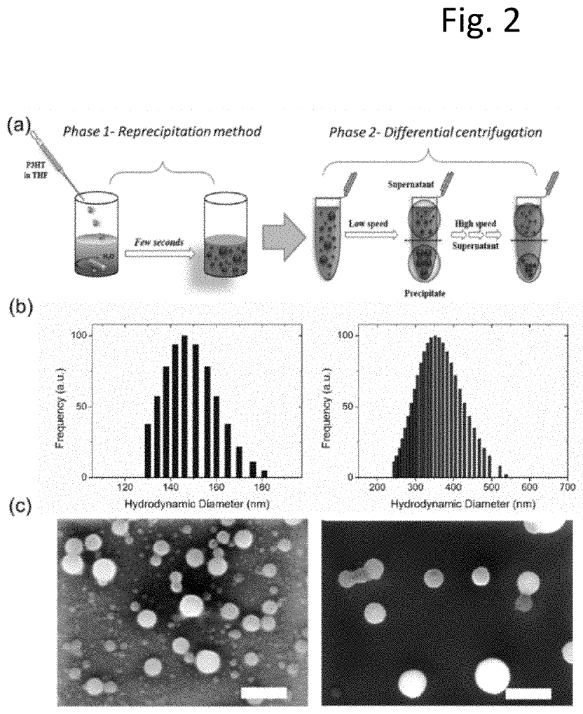

P3HT-NPs are prepared from pre-formed poly-(3-hexylthiophene). P3HT is synthesized by oxidative polymerization of 3-hexyl-thiophene with ferric chloride. Repeated preparations are highly reproducible in the characteristics of the polymer, namely regio-regularity (as estimated from ¹H-NMR), polydispersity, and spectroscopic features. The polymer displays good solubility in common organic solvents. The NPs are obtained from freshly prepared P3HT using the reprecipitation technique (solvent displacement method). No surfactants are employed and the whole process is carried out under sterile conditions using a laminar flow hood, sterilized laboratory glassware and water. Differential centrifugation, carried out employing sterilized centrifuge tubes, allows separating NPs into fractions of different size. Two fractions are selected, characterized by two different hydrodynamic diameters (HD, 148±18 nm and 344±60 nm) and low polydispersity indexes (PDI, 0.015 and 0.03 for 148 nm and 344 nm-sized NPs, respectively), which indicates a quite narrow particle size distribution, as determined by dynamic light scattering (DLS) measurements. Stability of P3HT-NPs is routinely assessed by Z-potential measurements. Solubility and stability in water are key-parameters for any targeted biological application. A major limitation of the most widely studied inorganic nanomaterials for bioimaging, such as inorganic quantum dots and magnetic NPs, is that they tend to be insoluble in water and require some kind of phase-transfer treatment or encapsulation. Similar issues have been partially solved in conjugated polymer NPs by covalent linking with ionic side groups, grafting on hydroxyl side groups, use of surfactants, encapsulation within micelles and loading with polyethylene glycol (PEG). In P3HT-NPs, values lower than −30 mV (−30.44 mV and −36.84 mV for 148 nm and 344 nm-sized NPs, respectively) are obtained by Z-potential measurements for both NPs populations, thus indicating intrinsically good stability in the aqueous environment. The control of NPs morphology is another, essential part of the preparation of particle suspension. In particular, synthesis of geometrically isotropic polymer micro- or submicron-spheres, with a well-defined surface topography, has been rarely reported so far, since conjugated polymers naturally tend to anisotropically crystallize, due to their rigid and planar backbone. NPs were cast on top of a silicon substrate and quickly dried by nitrogen. The isolated spots are due to individual NPs or small clusters generated by self-assembly process during the process of solvent evaporation. Opposite to DLS measurements, which are carried out in solution and thus include the contribution of the hydration shell of the NP, SEM measurements require dried samples and hence no additional hydration shell contributes. Accordingly, SEM yields a slightly shorter average particle size than DLS measurements. As expected, average spheres size is slightly lower than averaged values obtained by DLS, due to the absence of the hydration layer. Most importantly, one should appreciate the very well-defined topology and the surface smoothness, which are relevant also for other applications, such as the recently reported realization of polymer, fluorescent micro-resonators (FIG. 2).

Injection Procedure

In anesthetized rats, the conjunctiva is opened with scissors 1.5 mm from the limbus for about 2 clock hours in the upper quadrants. A 15° knife is use to perform a small incision (about 0.6 mm) through the sclera and the choroid 1 mm from the limbus. The retina is gently separated from the retinal pigment epithelium next to the incision injecting a small amount of viscoelastic material (high molecular weight hyaluronic acid). That permits to define clearly the sub-retinal space and find the right plane to introduce a 38-gauge needle connected to a syringe with the nanoparticles in vehicle solution. The position of the needle is critical. It is advisable to insert the needle for about 1.5 mm from the incision, tangentially to the sclera, in order to prevent any damage of the retina and the choroid. The suspension of nanoparticles is then injected. The tangential sub-retinal flow originated injecting with the needle in this position is very efficient in promoting a complete retinal detachment and a consequent uniform distribution of the nanomaterial. The scleral incision is coagulated with diathermy and the conjunctiva is gently repositioned over the small scleral wound. Subsequently, retina will spontaneously reattach to the choroid thanks to the osmotic pumping effect of the Retinal Pigment Epithelium (RPE).

Tolerability

Up to 2 months after the injection, the tolerability of the NPs is evaluated by using the following techniques:

(i) Ocular Coherence Tomography (OCT) in Live Animals.

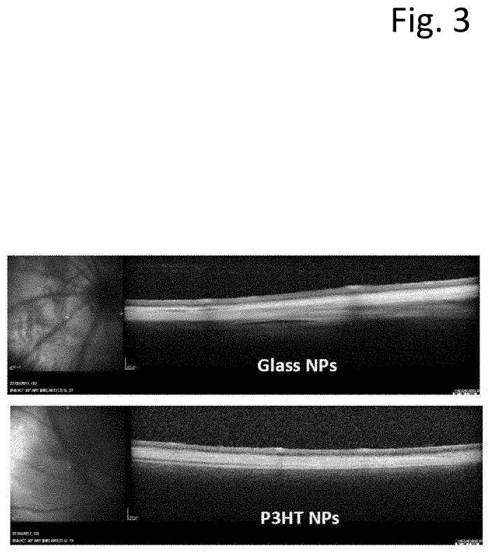

OCT is a non-contact imaging technique that provides high resolution scan of the retina, permitting to study the morphological features of the implanted eye, as well as the health state of the retina in contact with the NPs in live animals. By using this analysis, it has been found that the NP-injected retinas were in excellent shape, undistinguishable from non-injected retinas or the morphological features of the implanted eye as well as the health state of the retina in contact with the photoactive NPs in live animals (FIG. 3).

(ii) Retina Histology and Immunochemistry.

This kind of analyses were carried out on transverse retinal sections one month after NPs injection. Experimental animals are euthanized with CO₂. Enucleated eyes are fixed, cryoprotected and cryosectioned at 20 μm. NPs' fluorescence is observed in 3D using confocal microscopy in combination with bisbenzimide for nuclear staining and immunohistochemistry using markers for FGF (neurotrophic factor) and GFAP (glial proliferation) to monitor the inflammatory response to the implanted nanomaterials. The results indicate that, after a single sub retinal injection, NPs evenly distribute in the whole subretinal space, without altering retinal structure. The fact that the NPs diffuse from the site of injection to the whole subretinal surface but remain strictly confined to the external retina creates a uniform NP layer that takes the place of degenerate photoreceptors and covers the whole retina. At the same time, NPs do not interfere with the trophic and feeding of the residual photoreceptors by the retinal pigment epithelium.

Visual Rescue

The efficiency of bilaterally injected P3HT-NPs in promoting the rescue of visual functions in blind animals was evaluated one to two months after injection as follows:

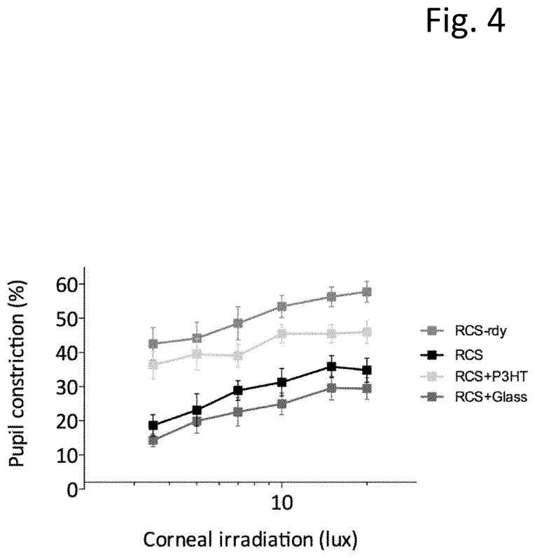

(i) Light-Evoked Pupillary Responses (FIG. 4).

To evaluate light sensitivity, pupillary responses to light beams of different intensities are measured in anesthetized animals. The pupil area is calculated with an infrared camera under dark conditions in response to increasing intensity of irradiance. NP-injected blind animals rescued their pupillary responses closely approaching the levels of control healthy animals, while blind animals injected with inert glass beads did not show any visual improvement.

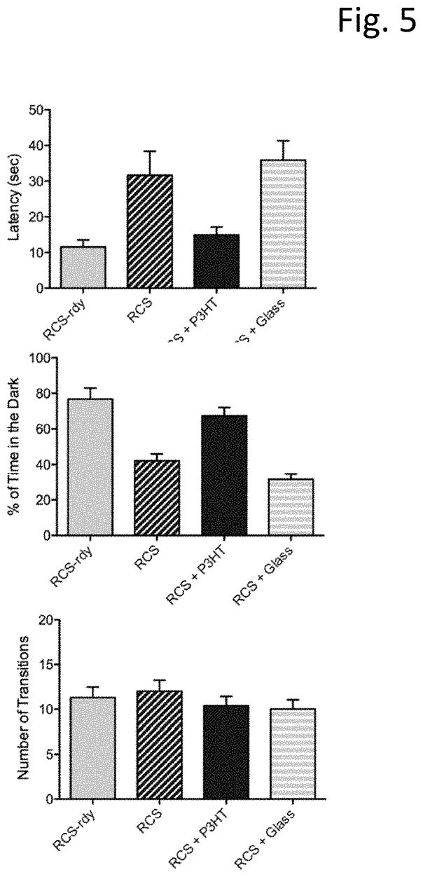

(ii) Visually Driven Behavior (FIG. 5).

To address whether the photoactive NPs have a behavioral impact in the experimental model light-dark box test has been used. This test is a light-driven behavioral analysis based on the innate aversion of nocturnal rodents to brightly illuminated areas. The experimental device consists of two communicating compartments, one illuminated and the other in total darkness. The test is performed in a dark room where animals are dark adapted for 30 minutes. After introducing the animal in the light compartment, a video is recorded to monitor the latency of escape from the illuminated area to the dark compartment and the percentage of time spent in each compartment. NP-injected blind RCS rats displayed latency to escape from light and percentages of time spent in the dark closely similar to the healthy controls in the absence of changes in the overall locomotor activity.

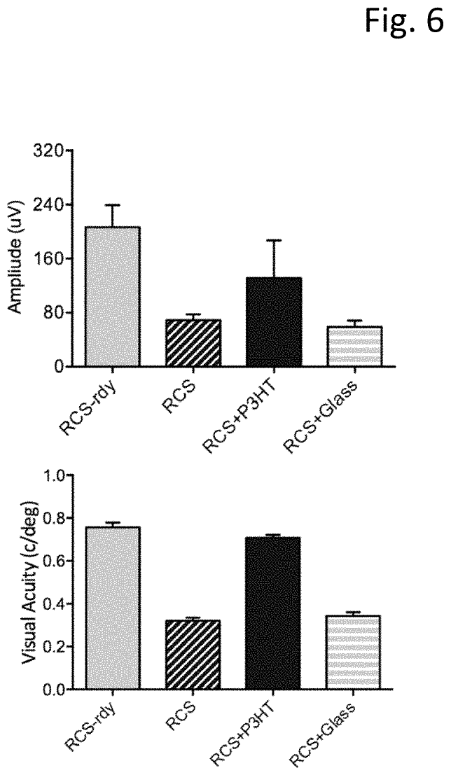

(iii) Full-field and patterned visually evoked potentials (ff-VEPs and pVEPs) in the Primary Visual Cortex (V1; FIG. 6).

The efficiency of photoactive NPs in restoring light-dependent responses in the visual cortex was evaluated in vivo by recording VEPs in anaesthetized animals. This technique allows assessing light sensitivity of cortical responses. Briefly, implanted animals were placed in a stereotaxic frame. A craniotomy in correspondence to V1 is performed and the dura gently removed. Then, a recording electrode is advanced at both superficial (100-150 μm) and deep (400-600 μm) layers of the primary visual cortex (V1). Electrophysiological recordings of VEPs were carried out by illuminating one eye and recording in the contralateral V1. NP-injected blind RCS rats displayed a significant rescue of the VEP amplitude with respect to healthy controls, while inert glass beads were ineffective.

(iv) Visual Acuity Analysis.

Visual stimuli were administered as horizontal sinusoidal gratings of increasing spatial frequencies (0.1 to 1 cycle per degree of visual angle) at 0.5 Hz on a monitor (20×22 cm area; 100% of contrast) positioned 20 cm from the rat's eyes and centered on the previously determined receptive fields. Visual acuity was then obtained by extrapolation to zero amplitude of the linear regression through the last four to five data points of VEP amplitude versus the log spatial frequency. NP-injected blind RCS rats displayed a complete rescue of the visual acuity to the same levels displayed by the non-dystrophic controls. The latter results indicate the efficacy of the injected NPs in increasing the high-resolution responses from the rat retina.

1. A method for treating a visual deficit of at least one eye of a subject in need thereof, comprising at least one step of injecting in the eye of said subject a therapeutically effective amount of photoactive nanoparticles (NPs).

2. A method for improving the spatial resolution of at least one eye of a subject in need thereof, comprising at least one step of injecting in the eye of said subject a therapeutically effective amount of photoactive nanoparticles (NPs).

3. The method according to claim 1, wherein the NPs are an aqueous dispersion of NPs.

4. The method according to claim 1, wherein the NPs comprise poly-(3-hexylthiophene).

5. The method according to claim 1, wherein the NPs have a diameter ranging from 50 to 450 nm.

6. The method according to claim 1, wherein the NPs have a polydispersity index (PDI) comprised between 0.008 and 0.05.

7. The method according to claim 1, wherein the NPs have a Z-potential value less or equal to −30 mV.

8. The method according to claim 1, wherein the NPs absorb the wavelength of visible light.

9. The method according to claim 1, wherein the NPs absorb the wavelength of the light ranging from 495 to 620 nm.

10. The method according to claim 1, wherein said NPs are administered intraorbitally by injection into a blood vessel that supplies blood to the eye.

11. The method according to claim 1, wherein said NPs are administered intraorbitally into the macula by first penetrating the sclera.

12. The method according to claim 1, wherein said NPs are administered intraorbitally by microinjection into a subretinal space.

13. The method according to claim 1, wherein said NPs are administered by microinjection into the subretinal space.

14. The method according to claim 1, wherein said NPs are administered by:
(i) Opening a conjunctiva of the eye to be treated,
(ii) Incising a sclera and a choroid of the eye to be treated,
(iii) Separating a retina from a retinal pigment epithelium of the eye to be treated,
(iv) Injecting a viscoelastic material into the retina;
(v) Injecting the NPs according to claim 1 in the sub-retinal space.

15. The method according to claim 14, wherein step (v) is performed by injecting the NPs tangentially to the sclera.

16. The method according to claim 1, wherein said visual deficit is associated with degeneration of the photoreceptors of the eye.

17. The method according to claim 16, wherein said degeneration is associated with a condition selected from: Retinitis Pigmentosa (RP) or related syndromes, preferably said syndromes being selected from: Usher Syndrome, Bardet-Biedl syndrome, Refsum disease, Batten disease, and Jalili syndrome; neuropathy, ataxia, NARP (neuropathy, ataxia, and retinitis pigmentosa) syndrome, inherited retinal degenerations, preferably elected from: Stargardt's disease and Leber's congenital amaurosis, and atrophic age-related macular degeneration (AMD).

18. The method according to claim 17, wherein said AMD is dry AMD.

19. The method according to claim 3, wherein before being administered the poly-(3-hexylthiophene) NPs is treated according to the following steps:
a) Collecting poly-(3-hexylthiophene), preferably by centrifugation;
b) Selecting NPs by different hydrodynamic diameter and by polydispersity index;
c) Suspending the NPs in aqueous solution;
d) Linking covalently the NPs with ionic side groups; and
e) Grafting on hydroxyl side groups.

20. The method according to claim 19, further comprising a step of encapsulating the NPs with micelles.

21. The method according to claim 19, further comprising a step of dispersing the NPs in polyethylene glycol (PEG).

22. A kit for performing the method according to claim 1 comprising the photoactive nanoparticles (NPs).

23. The kit according to claim 22, wherein the NPs comprise poly-(3-hexylthiophene).

24. The kit according to claim 22, wherein the NPs are an aqueous dispersion of NPs.

25. The kit according to claim 22, the NPs have a diameter ranging from 50 to 450 nm.

26. The kit according to claim 22 wherein the NPs have a polydispersity index (PDI) comprised between 0.008 and 0.05.

27. The kit according to claim 22, wherein the NPs have a Z-potential value less or equal to −30 mV.

28. The kit according to claim 22, wherein the NPs absorb the wavelength of visible light.

29. The kit according to claim 22, wherein the NPs absorb the wavelength of the light ranging from 495 to 620 nm.
